(19) United States
(12) Patent Application Publication
(10) Pub. No.: US 2008/0294058 A1
(43) Pub. Date: Nov. 27, 2008

(54) Wearable Device, System and Method for Measuring a Pulse While a User is in Motion

(76) Inventor: Dror Shklarski, Yavne (IL)

Correspondence Address: EMPK & Shiloh, LLP;c/o Landon IP, Inc., 1700 Diagonal Road, Suite 450, Alexandria, VA, 22314, US

(21) Appl. No.: 11/660,221

(22) PCT Filed: Aug. 10, 2005
(86) PCT No.: PCT/IL2005/000860

§ 371 Date: Jan. 20, 2008

Related U.S. Application Data
(60) Provisional application No. 60/601,637, filed on Aug. 16, 2004.

(51) Int. Cl. A61B 5/02

(52) U.S. Cl. 600/502

(57) ABSTRACT

A system, device and method are provided for monitoring a user's pulse, the system MAIN including a medical center server to provide commands to a wireless mobile pulse monitoring de-vice, the monitoring device including a pulse sensor sub-system including at least two pulse sensors adapted to accurately measure a pulse while a user is mobile.

### FIELD OF THE INVENTION

The present invention relates to wearable devices, systems and methods for monitoring and evaluating physiological and environmental parameters, and particularly to communication devices, systems and methods for measuring the pulse from the wearable device while the user is in motion.

### BACKGROUND OF THE INVENTION

Continuously monitoring a user's physiological condition generally requires the user's hospitalization, usually at great cost, especially where long term monitoring is required. In certain situations it is possible to monitor the physiology of users who are physically outside of the hospital, using wearable monitoring devices.

Wrist-worn devices have been developed to record a user's physiological data, such as the user's pulse and ECG, during a predetermined recording time. These devices may include event recorders that may capture a user's physiological data during a physiological “event”, such as a cardiac arrhythmia or an episode of user discomfort. The event recording may be activated manually by the user or automatically by determining when monitored physiological data meets predefined event criteria.

In particular, current state of the art systems for measuring the pulse from the wrist and/or the leg, using non-invasive techniques, typically use a few types of technologies, including: a) measuring at least 1-lead ECG and extracting the pulse form the duration between R-wave to R-wave. The 1-lead ECG may be taken, for example, from a chest belt with at least two electrodes and/or wearable device with one ECG sensor in the inner side and an additional sensor on top of it and the user is requested to put a finger of the other hand on the upper electrode; b) placing blood pressure devices on the wrist, that also measure the pulse using a cuff that is inflated and deflated. The aforementioned techniques typically require relatively large and bulky equipment (e.g., blood pressure with a cuff) and/or additional component/s in different parts of the body, e.g., chest belt. Common techniques may generally require the intervention of the user, e.g., the need to use both hands.

Another state of the art technique typically uses a piezoelectric sensor that translates pressure to an electric signal. If such a sensor is close enough to a vein it may enable detection of the pulse. But, this technique typically fails to measure the pulse while the wrist and/or the hand is mobile, since the sensor detects a lot of artifacts due to the motion and/or muscles tension.

### SUMMARY

According to some embodiments of the present invention, a system is provided for monitoring a user's pulse, the system including a medical center server to provide commands to a wireless mobile pulse monitoring device, the device including a pulse sensor sub-system including at least two pulse sensors adapted to accurately measure a pulse while a user is mobile.

According to additional embodiments of the present invention, a device for measuring pulse signals while a user is mobile is provided, the device including a pulse sensor sub-system including two or more pulse sensors adapted to accurately measure a pulse while the user is mobile; and a main controller to enable controlling of the pulse sensor sub-system.

According to further embodiments of the present invention, a method is provided for measuring the pulse of a subject using a wireless monitoring device, the method including: sensing of the subject's pulse using a pulse sensor sub-system within the wireless monitoring device, the pulse sensor sub-system including two or more pulse sensors; receiving pulse signals and noise signals from a first sensor in the pulse sensor sub-system, and receiving noise signals from a second sensor in the pulse sensor sub-system; digitizing the received signals; processing the signals; and generating a modified pulse signal.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic illustration of a medical monitoring and alert system according to some exemplary embodiments of the present invention;

FIGS. 2A, 2B, and 2C are schematic illustrations of external top, bottom, and side view layouts, respectively, of a wearable device according to some exemplary embodiments of the present invention;

FIG. 3 is a schematic illustration illustrating an internal layout of a wearable device according to some embodiments of the present invention;

FIG. 4 is a schematic illustration of an example of a pulse sensor sub-system included within a wearable device, according to some exemplary embodiments of the present invention; and

FIG. 5 is a flow chart illustrating a method for medical monitoring, according to some embodiments of the present invention.

### DETAILED DESCRIPTION OF THE INVENTION

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

Unless specifically stated otherwise, as apparent from the following discussions, it is appreciated that throughout the specification discussions utilizing terms such as “processing,” “computing,” “calculating,” “determining,” or the like, refer to the action and/or processes of a computer or computing system, or to a similar electronic computing device, that manipulates and/or transforms data represented as physical, such as electronic quantities within the computing system's registers and/or memories into other data similarly represented as physical quantities within the computing system's memories, registers or other such information storage, transmission or display devices. The processes and displays presented herein are not inherently related to any particular apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of embodiments of the invention as described herein.

It should be appreciated that according to some embodiments of the present invention, the method described below, may be implemented in machine-executable instructions. These instructions may be used to cause a general-purpose or special-purpose processor that is programmed with the instructions to perform the operations described. Alternatively, the operations may be performed by specific hardware that may contain hardwired logic for performing the operations, or by any combination of programmed computer components and custom hardware components.

Although the scope of the present invention is not limited in this respect, the wearable device disclosed herein may be implemented in any suitable wired or wireless device that may be a handheld, worn, or other suitable portable communications device. By way of example, the wearable devices may include wireless and cellular telephones, smart telephones, personal digital assistants (PDAs), wrist-worn devices, and other suitable wearable devices or any parts of them. Alternatively, according to other embodiments of the present invention, the system and method disclosed herein may be implemented in computers.

The present invention is directed to an improved wearable device, system, and method for remotely monitoring and/or measuring the pulse when a user is mobile, for example from the wrist and/or leg.

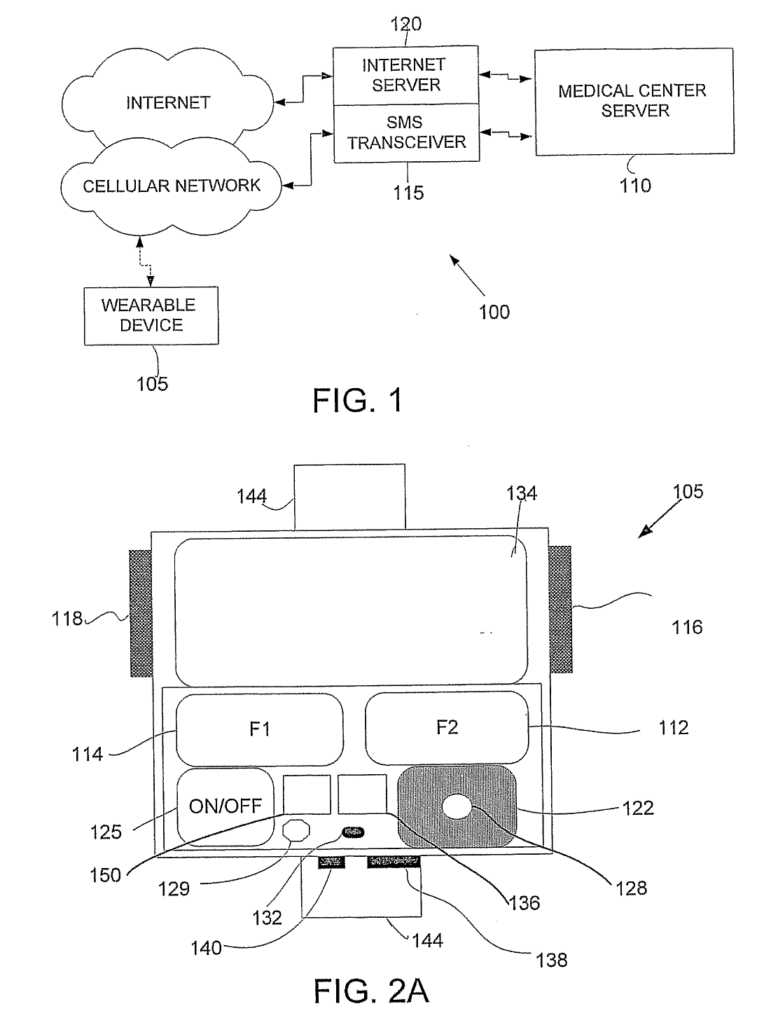

Reference is now made to FIG. 1, which schematically illustrates a medical monitoring and alert system 100 in accordance with some exemplary embodiments of the present invention. Medical monitoring and alert system 100 may include, for example, at least one wearable device 105 that may communicate with one or more medical center (MC) 110. The communication between wearable device 105 and MC server 110 may be, for example, wireless data communication, for example cellular communication technology (e.g., General Packet Radio Service (GPRS)), satellite communications technology, wireless LAN technology, WiFi, Bluetooth, ZigBee, or other suitable communications technologies, and a computer network, for example, the Internet or a local area network (LAN) etc. There may be a plurality of bi-directional and/or uni-directional communication channels between the MC server 110 and wearable device 105, and there may be a plurality of medical centers (MC) 110 and/or wearable devices 105 in a given embodiments of the present invention.

In one embodiment the bi-directional communication channel between the MC server 110 and wearable device 105 is a Short Message Service (SMS) channel that may enable communication of data via SMS transceiver 115 to and/or from the wearable device 105, via a cellular communications network. The SMS channel may enable transmission of messages from wearable device 105 to MC server 110, via SMS transceiver 115. In one embodiment the bi-directional communication channel between the MC server 110 and wearable device 105 is an Internet Protocol (IP) based channel, that may enable communication of data via Internet server 120, for example, using File Transfer Protocol (FTP) or other suitable data transfer protocols. In some embodiments a combination of communication, networks may be used. For example, if the SMS channel is not available and/or not chosen by the wearable device 105, wearable device 105 may communicate with MC server 110 using FTP. In other embodiments wearable device 105 may communicate with MC server 110 using, for example, SMS and Internet communications. In some embodiments wearable device 105 may communicate with MC server 110, via a Web interface, for example, a Website, where data, commands, and/or requests etc. may be entered and/or received by wearable device 105 and/or MC server 110.

In one embodiment the bi-directional communication channel between the MC server 110 and wearable device 105 may utilize TCP/IP protocol. In one embodiment a File Transfer Protocol (FTP) may be used to download new requests for physiological and/or environmental parameters measurements, e.g., measure the vital physiological parameters again, from MC server 110 to wearable device 105, and to upload data such as the results of such measurements from wearable device 105 to MC server 110. Usage of FTP or any other suitable protocol may require the wearable device 105 to logon as an FTP client to the Internet server 120.

In some embodiments a voice channel, as described below, may be used to enable the medical staff in MC server 110 to communicate with the user that is using wearable device 105 and/or vice versa.

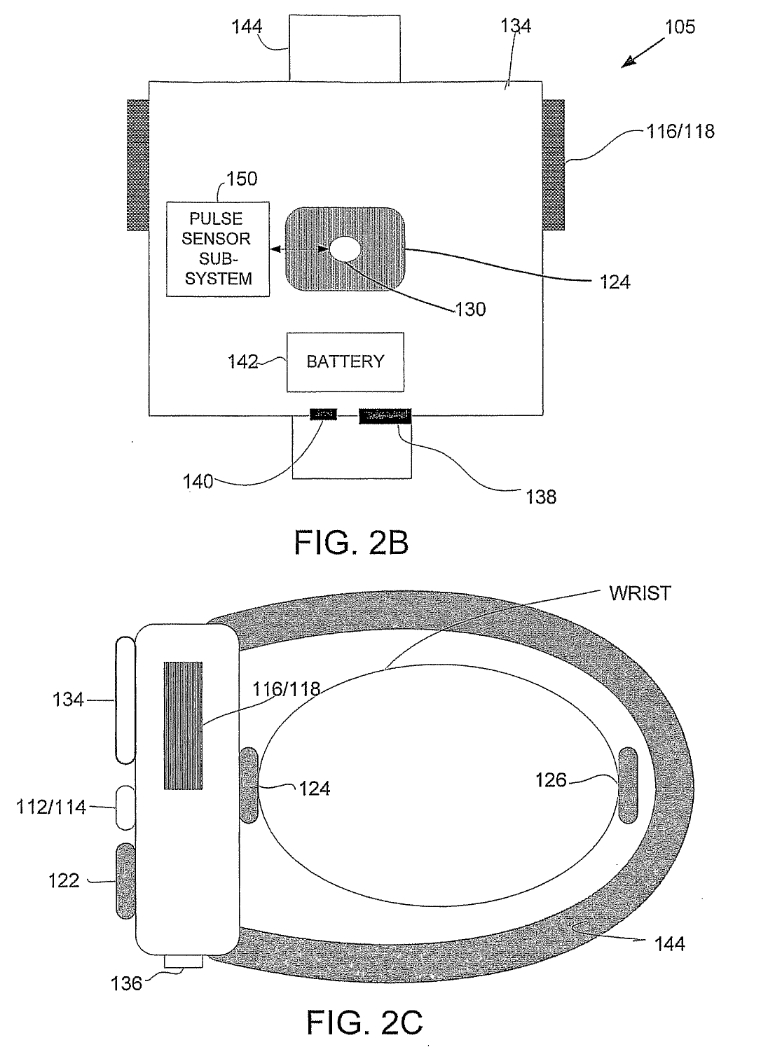

Reference is now made to FIGS. 2A, 2B, and 2C that schematically illustrate external top, bottom, and side view layouts, respectively, of a wearable device 105 in accordance with some embodiments of the present invention. Wearable device 105 may include, for example, input components such as functional buttons 112 and 114 for inputting data or commands to operate wearable device 105, emergency buttons 116 and 118, that may be used to manually initiate an emergency mode (e.g., by pressing them together or just pressing one of them), and an On/Off button 125 to switch wearable device 105 on or off. The On/Off button 125 may be unified with any of the other buttons, for example functional buttons 112 and 114. Wearable device 105 may include one or more electrodes, for example, an ECG RA (Right Arm) finger electrode 122, an ECG LA (Left Arm) wrist electrode 124 (FIG. 2B), and an ECG REF. (Reference) wrist electrode 126 (shown in FIG. 1C). Electrodes 122 and 124 may be located in any suitable locations on wearable device 105. For example, electrode 124 may be located on the top side of wearable device 105. In some embodiments the ECG REF. Wrist electrode 126 may be placed in any suitable location in the inner side of wearable device 105 or in the inner side of strap 144. Wearable device 105 may be worn on a user's left or right hand, or left or right foot, and the various components may be appropriately located to enable measuring of parameters whether on the left and/or right hand and/or foot.

In some embodiments ECG electrodes 124 and or 126 may be used to sense the ECG of the user, by, for example, performing ECG measurements when the user touches finger electrode 122 with his/her finger. In addition, wearable device 105 may include a blood oxygen saturation level (SpO₂) transceiver 127 and/or 128 to measure the level of the oxygen in the user's blood, one or more pulse transceivers 129 and/or 130 to measure the user's pulse, and/or a microphone 132 that may be used to enable the user's voice to be input, and optionally converted to electronic impulses for electronic communication. Blood oxygen level (SpO₂) transceiver 127 and/or 128 may be incorporated, for example, in the ECG RA finger electrode 122 and/or may be a separate sensor. Blood oxygen level (SpO₂) transceiver 127 and/or 128 may be located in a suitable location, for example, in the inner side of strip 144. In some embodiments wearable device 105 may include an alternative or additional pulse transceiver or sensor (e.g., 130) located in a suitable position in wearable device 105. In some embodiments, wearable device 105 may include one or more transceivers, electrodes, or sensors to enable measurement of blood pressure data, skin temperature data, respiration data, cardio impedance data, and/or other suitable data.

According to some embodiments of the present invention, wearable device 105 may include a pulse sensor sub-system 150, to enable accurate measuring of a user's pulse even when a user is mobile, as is described in detail below. Wearable device 105 may include a speaker 136 to enable a user to receive audio signals, for example voice communication, from MC server 110. When wearable device 105 is operated in continuous mode, wearable device 105 may, for example, continuously or according to a pre-defined schedule, read the pulse of the user, for example using the pulse transceiver(s) 129,130. Pulse transceivers 129 and 130 may be incorporated, for example, within electrode 124 or may be separate from electrode 124. The pulse and/or other parameters may be presented on the display area 134 of wearable device 105. The pulse and/or other parameters may be transferred to the MC server 110. Other sensor mechanisms may be used.

Wearable device 105 may include a display area 134, to display additional information such as, for example, medical parameters of the user, messages received from a medical center (MC), operational instructions, date and time, parameters that are related to functional elements of wearable device 105 etc. Display area 134 may be, for example, a colored display and/or a monochromatic one. Display area 134 may be, for example, interactive or be touch sensitive, or voice sensitive, or display any combination of alphanumeric characters, and/or text and/or 2 dimensional and/or 3 dimensional graphics and/or icons.

Additional elements in wearable device 105 may include one or more service connector, for example service connector 138 that may connect the wearable device 105 to external units such as, for example, a computer or a testing unit or an external medical device, or display unit, or communication unit. Wearable device 105 may include a charge connector 140 that may be used to connect wearable device 105 to a power source to enable charging of a battery 142 (FIG. 2B). A charger connector 140 may be included in service connector 138. Wearable device 105 may include strap 144 that may be used to attach wearable device 105 to the wrist or other location of the user. Wearable device 105 may include various other suitable components and/or devices, which may be implemented using any suitable combination of hardware and/or software.

In accordance with some embodiments of the present invention, medical monitoring and alert system 100 may operate in at least one of keeper mode, extended mode, and emergency mode, as described below.

The keeper mode may be used as the default mode of wearable device 105, such that wearable device 105 may enter this mode when the device is switched on. Other modes may alternatively be used as the default mode. In the keeper mode, wearable device 105 may, for example, continuously or intermittently, read the pulse and/or another parameters of a patient. In this mode, wearable device 105 may display parameter data on display area 134, may alert the patient with a message on display area 134, and/or may alert the patient using an audible signal via speaker 136, for example, by playing back predefined audio signals. In addition, wearable device 105 may transmit the measured parameters and/or results from analyses or processing of the measured parameters, to MC server 110, for example, using an FTP channel and/or SMS channel. In the event where the medical staff in MC server 110 determines that the user's pulse is abnormal, according to predetermined criteria or ranges described in detail below, wearable device 105 may alert the user. According to some embodiments of the of the present invention wearable device 105 may determine when one or more parameters are abnormal or, for example, in a danger range, instead of or in addition to the medical staff in MC server 110. According to some embodiments of the of the present invention MC server 110 may automatically determine when one or more parameters are abnormal or, for example, in a danger range, instead of or in addition to the medical staff in MC server 110. Additionally, wearable device 105 may send a warning message to MC server 110, using, for example, the SMS channel, FTP channel etc. When wearable device 105 is operated in keeper mode, physiological parameters and/or vital signs such as pulse, SpO₂, and ECG may be monitored at selected intervals, for example, every twelve hours.

In the extended mode, wearable device 105 may be set to perform operations according to a pre-defined schedule to perform, for example, to periodically measure oxygen levels in the patient's blood (SpO₂) and/or ECG. In this mode, wearable device 105 may display parameter data on display area 134, may alert the user with a message on display area 134, and/or may alert the user using an audible signal via speaker 136, for example, by playing back predefined audio signals. In addition, wearable device 105 may transmit the measured parameters and/or results from analyses or processing of the measured parameters, to MC server 110, for example, using FTP channel and/or SMS channel. When wearable device 105 is operated in extended mode, physiological parameters and/or environmental and/or vital signs such as pulse, SpO₂, and ECG, may be monitored, for example, five times a day by default (e.g., the default may be determined at shorter or longer intervals, in relation to the situation in Keeper mode). If the medical staff at MC server 110 detect or the MC server 110 automatically detects, for example, that the heart rate, oxygen level in the blood, and/or ECG records and/or other data are abnormal (e.g., according to pre-defined criteria or ranges as discussed below), wearable device 105 may alert the user by providing output signals in the display area 134 or via speaker 136. Additionally or alternatively, wearable device 105 may send a message to MC server 110 or to another destination, for example, using the FTP channel.

In emergency mode a user may initiate operation of the medical monitoring and alert system 100 by pressing, for example, any of the emergency buttons 116 or 118. When operating in emergency mode, wearable device 105 may send emergency messages to MC server 110 or to another destination using, for example, the FTP channel. Emergency messages may additionally or alternatively be sent to MC server 110 or to another destination, via the SMS channel, for example, in cases where the FTP channel is not available. In addition, when entering an emergency mode, measurement of SpO₂ and ECG levels, and/or alternative or additional parameters, may be initiated. The medical staff of MC server 110 or the MC server 110 itself may initiate a call to the user of wearable device 105, or may send a message etc.

According to some embodiments of the present invention requests for new pulse measurements (outside the regular measurements based on the modes of the device) may be implemented to enable the medical staff in the medical center and/or the medical center system itself to request additional measurements of physiological parameters and/or environmental and/or vital signs and thus to control the operation of device 105. The new request/s may be sent directly into wearable device 105 using wireless data communication, and/or may be remotely transferred to wearable device 105. In this way the measurement may be executed, optionally remotely, by MC server 110, at the discretion of the medical staff. For example, the MC may remotely initiate a new ECG measurement for wearable device 105, optionally for each individual user.

In accordance with some embodiments of the present invention, wearable device 105 may be able to receive SMS messages from the MC server 110 via the SMS channel. The SMS messages may be displayed to the user on display area 134. The SMS messages may be selected from a list of pre-defined messages or written by the medical staff in MC server 110. SMS messages may include instructions to perform additional tests, embedded or attached software updates, instructions to logon to Internet server 120 for receiving new sets of requested measurements, instructions to go to the MC, updated medical parameters or diagnostic ranges, or other suitable data.

In some embodiments of the present invention, new measurements may be defined for an individual user, to facilitate the monitoring and/or analysis of the sensed physiological parameters and/or environmental and/or vital signs of wearable device 105. For example, the medical staff in MC server 110 or the MC server 110 itself or the technical staff of the MC server 110 may initiate diagnostic changes to help determine a user's status, for example enabling remote testing of the user's physiological parameters and/or environmental and/or vital signs etc. New ranges, commands etc. may be determined for each user by the medical staff, and may be programmed into the wearable device 105 by wireless data communications.

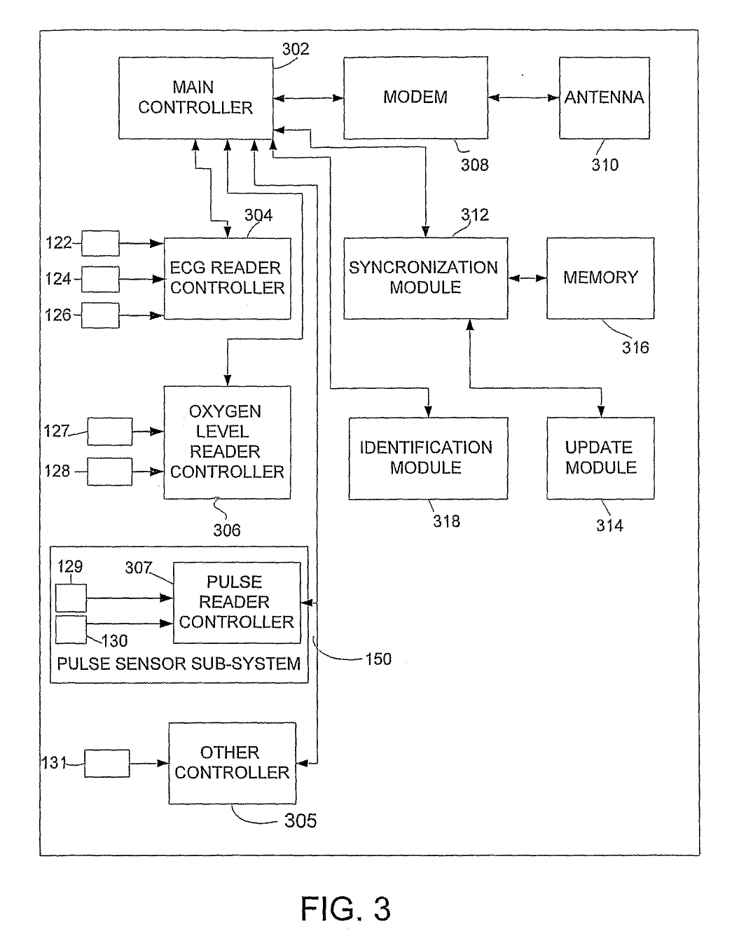

Reference is now made to FIG. 3, which is a schematic illustration of an internal layout of wearable device 105 in accordance with some embodiments of the present invention. Wearable device 105 may include, for example, a main controller 302 to control wearable device operation. Wearable device 105 may include an ECG controller 304 that may receive input from, for example, ECG electrodes 122, 124, and/or 126 (also shown in FIGS. 2A, 2B, and 2C, respectively), or from other sensors or combinations of sensors, and may generate output signals through main controller 302. Wearable device 105 may include any blood oxygen level controller 306 that may receive input from, for example, the SpO₂ transceiver 127 and/or 128, or from other sensors or combinations of sensors, and may generate output signals through main controller 302.

Wearable device 105 may include a pulse level controller 307 that may receive input from two or more pulse sensors 129 and 130, or from other transceivers or sensors, or combinations of transceivers or sensors, and may generate suitable output signals through main controller 302. Pulse sensors 129 and 130 may be located at two or more suitable locations on a user's body, for example, at suitable locations in proximity to the user's wrist, neck, temple, groin, behind the knees, or on top of the foot, or in other suitable locations. Wearable device 105 may utilize pulse level controller 307 to operate pulse sensors 129 and 130 to measure the pulse and/or heart rate (for hereinafter it is named pulse) continuously and/or non-continuously for any duration of time and/or single measurement and/or any combination of single measurement etc. The duration and/or interval of measurement may take few seconds to few tens of seconds or more. In some embodiments of the invention the interval of measuring may be a sliding window interval and then a new pulse measurement may be received beat by beat.

Wearable device 105 may include pulse sensor sub-system 150, as is described in detail below, to enable measurement of a pulse without any limitation on the movements and/or non-movements of the user of the wearable device. For example, a user's pulse may be accurately measured during general body motion and/or specific motion of a body part of limb etc. Pulse sensor sub-system 150 may be adapted to measure the pulse while the body and/or any part of it is moving intentionally and/or non-intentionally. For example, while part of the body may or may not move, e.g., sitting, standing, walking, climbing and/or descending stairs, eating, reading a book, working on a computer, playing the piano, etc. Other examples of movements of the body while pulse sensor sub-system 150 may measure the pulse may be during driving and/or sitting in a car (the car may be shaky from time to time), using a bus or an underground train, sailing in a boat etc. In some embodiments of the invention, pulse sensor sub-system 150 may measure the pulse while the user of the wearable device 105 is stationary and/or not moving his/hers wrist and/or hand and/or leg. In other embodiments of the invention, the pulse sensor sub-system measures may measure the pulse while there is tension in the muscles where the wearable device is being worn, for example while carrying a baggage/bag in the hand while the device is worn in the same hand. In other embodiments pulse sensor sub-system 150 may identify situations where it cannot measure reliable pulse measurement due, for examples to exceptional noise or artifacts, and it may report such situations, or alert a user, for example, to periodically measure their pulse using other means. Other sensor(s) 131 and controller(s) 305 may be used.

Wearable device 105 may further include at least one modem 308, to transmit and receive data to and from MC server 110, and at least one antenna 310. Wearable device 105 may include one or more of synchronization module 312, update module 314, memory 316, and identification module 318. Identification module 318 may include, for example, a Subscriber Identity Module (SIM) card and/or alternative identification means.

In some embodiments, main controller 302 may receive data from input components, for example, data received from functional buttons 112 and 114, emergency buttons 116 and 118, On/Off button 125, and/or from other components, such as service connector 138, charge connector 140, and battery 142. Main controller 302 may generate output that may be transferred to output components, for example display area 134, modem 308, antenna 310 etc.

In some embodiments, ECG controller 304 may receive signals indicative of physiological parameters and/or environmental and/or vital signs of the user from ECG RA finger electrode 122, ECG LA wrist electrode 124, and/or ECG REF wrist electrode 126. ECG controller 304 may receive data, for example via main controller 302, from functionality buttons 112 and 114, emergency buttons 116 and 118, or other suitable sources. ECG controller 304 may transfer data, for example via main controller 302, to output components, for example screen display 134, speaker 136, modem 308 etc. In some embodiments, Oxygen Level controller 306 may receive signals indicative of physiological parameters and/or environmental and/or vital signs of the user from sensors 127 and/or 128. Oxygen Level controller 306 may receive data, for example via main controller 302, from functionality buttons 112 and 114, emergency buttons 116 and 118, or other suitable sources. Oxygen Level controller 306 may transfer data, for example via main controller 302, to output components, for example screen display 134, speaker 136, modem 308 etc.

In some embodiments, Pulse Level controller 307 may receive signals indicative of physiological parameters and/or environmental and/or vital signs of the user from electrodes 129 and 130, or other suitable transceivers or sensors. Pulse Level controller 307 may receive data, for example via main controller 302, from functionality buttons 112 and 114, emergency buttons 116 and 118, or other suitable sources. Pulse Level controller 307 may transfer data, for example via main controller 302, to output components, for example screen display 134, speaker 136, modem 308 etc.

In some embodiments, the main controller 302, ECG controller 304, Oxygen level controller 306 and pulse reader controller 307, as well as other controllers, for example for blood pressure, for blood sugar level, etc. may be implemented as in a single controller or in multiple separate or combinations of controllers.

In some embodiments, wearable device 105 may include sensors and controllers to enable measurement and usage of blood pressure data, skin temperature data, body temperature data, respiration data, cardio impedance data, and other suitable data. Respective controllers may receive signals indicative of physiological parameters and/or environmental and/or vital signs of the user from respective sensors. Respective controllers may receive data, for example via main controller 302, from functionality buttons 112 and 114, emergency buttons 116 and 118, or other suitable sources. Respective controllers may transfer data, for example via main controller 302, to output components, for example screen display 134, speaker 136, modem 308 etc.

In accordance with some embodiments of the present invention, wearable device 105 may start the vital parameter measurement after their validity has been checked. In other embodiments wearable device 105 may postpone the measurement.

In accordance with some embodiments of the present invention, wearable device 105 may inform the user through one or more output components, for example display area 134 and/or speaker 136, when a new pulse measurement was done and/or while it is being done and/or whether the pulse measurement is valid.

In some embodiments of the present invention, oxygen level controller 306 may receive signals received from SpO₂ transceiver 127 and/or 128, and may receive data and signals received by main controller 302 from function buttons 112 and 114, and emergency buttons 116 and 118 etc. Oxygen level controller 306 may generate output signals that may be transferred via main controller 302 to one or more output components of wearable device 105 such as screen display 134, and to modem 308 to transfer the data regarding the oxygen level in the blood of the user or its pulse to MC server 110.

In some embodiments of the present invention, modem 308 may transfer and receive data from and to a MC server 110, for example, via antenna 310. For example, modem 308 may receive instructions sent from MC server 110 through the SMS channel, or answer to voice calls received from MC server 110. Modem 308 may download new software updates, for example including updated medical parameters, and updated diagnostic ranges, etc. Modem 308 may receive data, for example sensed measurements of physiological parameters and/or environmental and/or vital signs, from main controller 302. Modem 308 may receive and transfer signals from and to microphone 132, identification module 318, and speaker 136. Modem 308 may be a wireless modem, or another suitable technology for enabling data transmission from or to wearable device 105.

In some embodiments of the present invention, data and signals transferred between the components and modules of wearable device 105 may be transferred in serial communication lines, I/O lines, and/or designated lines. For example, a V_{BAT} signal may activate an alert indicating that battery 142 is weak, and a V_{CHARGER} signal may activate an alert indicating that battery 142 is charged.

In some embodiments, synchronization module 312 may receive data from various components in wearable device 105, and may synchronize the data before transferring it to main controller 302. For example, synchronization module 312 may receive data from update module 314, memory unit 316 and/or identification module 318, and may determine, for example, which data is the most updated, and may initiate transfer of the most updated received data to main controller 302.

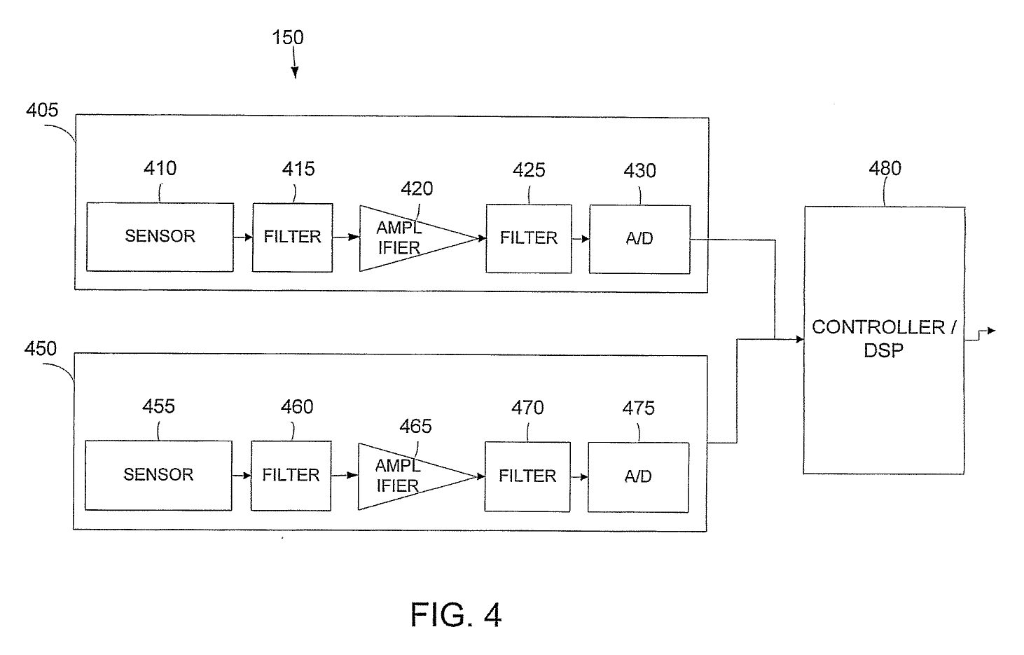

Reference is now made to FIG. 4, which schematically illustrates an example of pulse sensor sub-system 150, in accordance with some embodiments of the present invention. The present invention may help deal with the problem of the artifacts and/or noise and may enable substantially accurate measurement of the pulse in resting and non-resting situations. In accordance with some embodiments of the present invention pulse sensor sub-system 150 may include, for example, two or more sensors 410 and 455 and suitable electric circuitry. Sensors 410 and 455 may enable sensing of pulse signals, and channeling or processing of these signals in blocks, channels or circuits 405 and 450, respectively. Sensors 410 and 455 may be piezoelectric and/or optical sensors, or other suitable sensors. Sensors 410 and 455 may be included within sensors 129 and 130 of FIG. 3, or may be independent of sensors 129 and 130. Block or circuit 405 may be designed to measure the pulse signals and the noise signals and/or artifacts (e.g., unwanted signals), while block or circuit 450 may be designed to substantially measure the noise signal and/or the artifacts. It is to be noted that the artifacts and/or noise measured/received by both channels 405 and 450 may not have the exact characteristics (e.g., amplitude, power spectrum, delay between the noise/artifacts appearing in the two channels). Differences between the noise/artifact characteristics received by the channels may vary all the time and/or from time to time. These and other relevant effects may be included in the configuration of DSP 480, to help in processing the modified pulse measurement. Circuits 405 and 450 may channel signals to a controller or Digital Signal Processor (DSP) 480, to enable pulse sensor sub-system 150 to extract the pulse measurement. DSP 480 may be may be included within pulse reader controller (307 of FIG. 3), or may be independent of controller 307.

Sensor 410 may be required to be in proximity with a part of the body where the pulse may be detected, for example the arm, leg and/or other suitable area, to detect the pulse and the artifacts. The proximity may require contact with the user's skin, and may be positioned tightly and/or loosely on the selected area. In some embodiments of the present invention the intensity of the tightness/looseness of the sensor to the body may be changed during the measurements and/or between the measurements. In other embodiments of the invention the sensor(s) may not be in direct contact with the body, for example, it may be in contact with the enclosure of the wearable device 105. Sensor 410 may be connected, for example, to the enclosure of wearable device 105, relatively far from the body of the user compared to sensor 455, to enable detection of the artifact (e.g., to primarily or substantially measure the artifacts). Sensor 455 may be required to be in proximity with a part of the body where artifacts may be detected, for example any suitable area where selected artifacts may be measured. In some embodiments sensor 455 may be placed separately from the users body, for example on an automobile chair, to sense a predominant source of noise when the user is traveling in an automobile.

Each of sensors 410 and 455 may share the same electronic circuitry, or may have separate or partially separate circuitry. In some embodiments sensors 410 and 455 may be connected to respective analogue filters 415 and 460. The characteristic of these filters may be identical and/or different from channel to channel. In some embodiments of the invention the characteristic of the filters may be changed dynamically by DSP 480, together or individually.

In some embodiments signals may be channeled via amplifiers 420 and 465 respectively, to amplify the signals from sensors 410 and 455. The characteristic of these amplifiers may be identical and/or different from channel to channel. In some embodiments of the invention the characteristic of the filters may be changed dynamically by DSP 480, together or individually.

In some embodiments signals may be passed through additional filters 425 and 470 respectively. The characteristic of these filters may be identical and/or different from channel to channel. In some embodiments of the invention the characteristic of the filters may be changed dynamically by DSP 480, together or individually. In some embodiments signals may be converted from analogue to digital using converters 430 and 475 respectively. The characteristic of these converters may be identical and/or different from channel to channel. In some embodiments of the invention the characteristic of the converters may be changed dynamically by DSP 480, together or individually. The sample rate of A/D converters 430 and 475 may be, for example, two or more times higher than the pulse rate (e.g., two times the maximum measured possible pulse/divided by 60 sec), which may include, for example, all signals from sensors 410 and 455. The sampling rate may be higher in order to include 2^{nd}, 3^{rd} and higher harmonies of the pulse and artifacts, to ease the processing inside DSP 480.

At both blocks 405 and 450 the parameters of each channel may take into account that the signal may not be saturated before entering A/D converters 430 and 475 respectively. Moreover, converters 430 and 475 may use a high number of bits per sample (e.g., above 12 bits) in order to provide a good resolution between the sampling in each channel. In some embodiments A/D converters 430 and 475 may be part of DSP 480.

In accordance with some embodiments of the present invention, blocks 415, 425 460 and/or 470 may be extracted out of the scheme of pulse sensor sub-system 150. According other embodiments of the invention the use of the amplifiers 465 and/or 420 may be waived in one block 405 or 450 or in both blocks.

DSP 480, which may be the computing system of pulse sensor sub-system 150, may be responsible to use both channels 405 and 450 in order to remove or reduce the effect of the artifacts to a level that the pulse of the user can be extracted. The output of DSP 480 may be the pulse of the user, and this output may contain additional information, for example, the quality of the measurement, validity of the measurement, the results of internal tests of sub-system 150 etc. The output may be communicated in any format or standard and/or protocol (e.g.: RS-232, USB). DSP 480 may also be responsible for the parameters of elements 410, 415, 420, 425, 430, 455, 460, 465, 470, 475 and/or 480. DSP 480 may be used to process signals from one or more channels or circuits. In some embodiments multiple DSPs may be used. DSP 480 and/or other controllers (e.g., main controller 302) may enable changing of the parameters of one or more circuits or channels at selected time intervals, for example, to configure the parameters equal to each other or different from each other. In some embodiments DSP 480 may be implemented in a low power consumption component, thus the all power consumption of sensor pulse sub-system 150 may be less than few miliampers.

In accordance with some embodiments of the present invention, DSP 480 may use several techniques in order to do provide a modified pulse measurement, for example, a pulse measurement substantially without associated noise or artifacts. One of the techniques may be by analyzing the signals received from both channels 405 and 450 in the time domain. While using the time domain analysis DSP 480 may take into account that similar artifacts are collected at different amplitudes. DSP 480 may use analysis techniques in the frequency domain, for example analyzing the spectrum of the signals received from channels 405 and 450. DSP 480 may use several techniques to transfer the signal from the time domain to the frequency domain, for example, using the Fast Fourier Transform.

According to some embodiments of the present invention the analysis of the signal within DSP 480 may be done using one or more of the following techniques: filtering the signals (e.g., Chebyshev filter 1^{st} order or 2^{nd} order or higher order, Butterworth filter in any order, Bessel filter in any order); adapting the parameter/s of the filter/s (e.g., using adaptive filters); analyzing the signals using a window of samples in different intervals and/or a fix interval (e.g., 30 second intervals, 10 second intervals, 120 second intervals etc.); using sliding windows of samples; and controlling of the parameters of the analogue circuits at channels 405 and 450 etc. The techniques may be changed by DSP 480 at selected time intervals, and/or may stay the same for selected periods of time. The DSP 480 may combine two or more processing techniques, for example, working in the time domain to reduce the noise and artifacts in the pulse signal (channel 405), and then resuming the reduction using techniques in the frequency domain.

In accordance with some embodiments of the present invention MC server 110 may initiate an authentication process, when wearable device 105 approaches or connects to a computer associated with MC server 110, to download a request for a new measurement. For example, a Secure Sockets Layer (SSL) session or other suitable methods may be used to authenticate the data communication between MC server 110 and wearable device 105. In accordance with some embodiments of the present invention data privacy may be enabled by using authentication and/or encryption technologies.

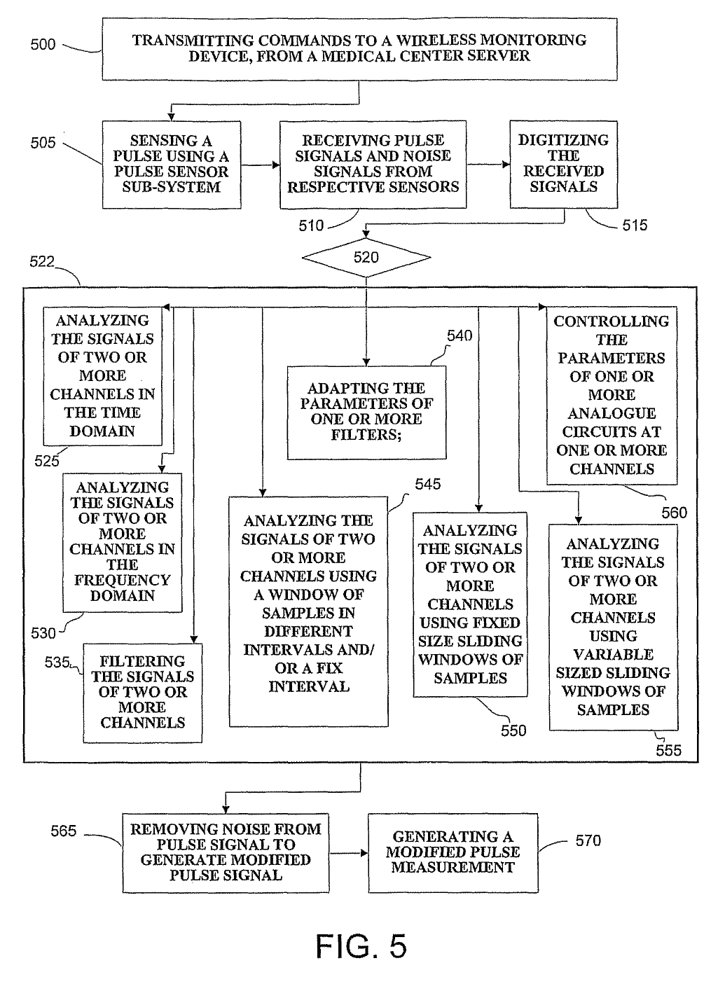

Reference is now made to FIG. 5, which is a flow chart illustrating an example of a method of pulse signal measuring, in accordance with some embodiments of the present invention. At block 500 commands may be transmitted to a wireless monitoring device, for example from a medical center server or other selected source, to measure a pulse signal. In some embodiments the initiator of the pulse measurement may be the user and/or the monitoring device, based on a pre-determined schedule and/or based on previous measurement(s). At block 505 the pulse sensor sub-system may measure a user's pulse using two or more sensors, a first sensor to substantially measure pulse signals and noise signals, and a second sensor to substantially measure noise signals. At block 515 both pulse and noise signals (e.g., artifacts) received or measured by the sensors may be processed, the processing of the signals being initiated, at block 515, by digitizing the received signals. At block 520 a decision may be made, automatically, how to process the received signals. In some embodiments the decision of how to process the signals may be made remotely, for example, by a medical center.

For example, at block 522 one or more steps, and/or any combination of steps may be implemented to process the digitized signals, by the pulse sensor sub-system. According to some embodiments of the present invention, at block 525, the signals of two or more channels may be analyzed in the time domain; at block 530, the signals of two or more channels may be analyzed in the frequency domain; at block 535, the signals of two or more channels may be filtered, by one or more filters; at block 540, the parameters of one or more filters may be adapted; at block 545, the signals of two or more channels may be analyzed using a window of samples in different intervals and/or a fix interval; at block 550, the signals of two or more channels may be analyzed using fixed size sliding windows of samples; at block 555, the signals of two or more channels may be analyzed using variable sized sliding windows of samples; and at block 560, the parameters of one or more analogue circuits may be controlled at one or more channels. One or more of the above steps may be implemented, or any combinations of steps may be implemented. At block 565 the noise signals or artifacts may be removed from the pulse signal thus generating or providing a modified pulse signal, for example, with less, minimal or negligible noise. At block 570 the modified pulse signal may be used to calculate and/or generate the modified pulse measurement or value.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

What is claimed is:

1. A system for monitoring a user's pulse, comprising a medical center server to provide commands to a wireless mobile pulse monitoring device, said device including a pulse sensor sub-system including at least two pulse sensors adapted to accurately measure a pulse while a user is mobile.

2. The system of claim 1, wherein said pulse sensor sub-system includes a sensor, an amplifier, and an analog-to-digital converter.

3. The system of claim 1, wherein said pulse sensor sub-system includes a Digital Signal Processing unit.

4. The system of claim 1, wherein said pulse sensor sub-system includes one or more analog filters.

5. The system of claim 1, wherein the functioning of said pulse sensor sub-system may be remotely configured by said server.

6. The system of claim 1, comprising an array of sensors for measuring physiological parameters.

7. The system of claim 1, comprising an array of sensors for measuring environmental parameters.

8. The system of claim 1, comprising an array of sensors for measuring physiological parameters and environmental parameters.

9. The system of claim 1, comprising one or more sensors selected from the group consisting of piezoelectric sensors and optical sensors.

10. The system of claim 1, wherein said monitoring device is to function in one or more of keeper mode, extended mode, and emergency mode.

11. The system of claim 1, wherein said monitoring device is to measure one or more selected parameters continuously and/or intermittently.

12. The system of claim 1, wherein said monitoring device is to automatically send a warning message to said medical center server if parameters measured exceed a selected threshold.

13. The system of claim 1, wherein said remote configuration of said mobile monitoring device includes remotely implementing selected software update(s).

14. The system of claim 1, wherein communications between said monitoring device and said medical center are encrypted.

15. The system of claim 1, wherein communications between said monitoring device and said medical center are authenticated.

16. The system of claim 1, wherein communications between said monitoring device and said medical center are encrypted and authenticated.

17. The system of claim 1, comprising a synchronization module.

18. The system of claim 1, comprising an update module.

19. The system of claim 1, comprising a data encryption module and a data authentication module.

20. A device for measuring pulse signals while a user is mobile, the device comprising:
a pulse sensor sub-system including two or more pulse sensors adapted to accurately measure a pulse while the user is mobile; and
a main controller to enable controlling of said pulse sensor sub-system.

21. The device of claim 20, wherein said pulse sensor sub-system includes at least one sensor to substantially measure pulse signals and at least one sensor to substantially measure pulse artifact signals.

22. The device of claim 20, comprising one or more sensors selected from the group consisting of an ECG sensor, Oxygen level sensor, pulse sensor, blood pressure sensor, SpA₂ sensor, glucose level sensor, sweat sensor, skin temperature sensor, pH level sensor, external temperature sensor, air humidity level sensor, and pollution level sensor.

23. The device of claim 20, comprising one or more modules selected from the group consisting of synchronization module, update module, data encryption module, data authentication module, data encryption module, and data authentication module.

24. A method for measuring the pulse of a subject using a wireless monitoring device, the method comprising:
sensing of the subject's pulse using a pulse sensor sub-system within the wireless monitoring device, said pulse sensor sub-system including two or more pulse sensors;
receiving pulse signals and noise signals from a first sensor in said pulse sensor sub-system, and receiving noise signals from a second sensor in said pulse sensor sub-system;
digitizing the received signals;
processing said signals; and
generating a modified pulse signal.

25. The method of claim 24, comprising using said modified pulse signal to generate a modified pulse measurement.

26. The method of claim 24, comprising transmitting commands to a wireless monitoring device, from a medical center server, to measure a pulse.

27. The method of claim 24, comprising removing said noise signals.

28. The method of claim 24, wherein said processing includes analyzing the signals of two or more channels in the time domain.

29. The method of claim 24, wherein said processing includes analyzing the signals of two or more channels in the frequency domain.

30. The method of claim 24, wherein said processing includes analyzing the signals of two or more channels in the time domain followed by analyzing the signals of two or more channels in the frequency domain.

31. The method of claim 24, wherein said processing includes filtering the signals of two or more channels.

32. The method of claim 24, wherein said processing includes adapting the parameters of one or more filters.

33. The method of claim 24, wherein said processing includes analyzing the signals of two or more channels using a window of samples in different intervals and/or a fix interval.

34. The method of claim 24, wherein said processing includes analyzing the signals of two or more channels using sliding windows of samples.

35. The method of claim 24, wherein said processing includes controlling the parameters of one or more analogue circuits at one or more channels.

36. The method of claim 24, wherein said processing includes checking the validity of said commands.

37. The method of claim 24, wherein said processing includes one or more techniques to analyze the signals of two or more channels, the techniques selected from the group consisting of analyzing in the time domain, analyzing in the frequency domain, filtering the signals, usage a window of samples in different intervals and/or a fix interval, and usage of sliding windows of samples.

38. The method of claim 37 wherein said techniques are adaptive from a first measurement to a second measurement.

39. The method of claim 37 while said techniques are adaptive from a first sample of signals of two or more channels to a second sample of signals of two or more channels.

40. The method of claim 24, wherein said pulse sensor sub-system includes an array of sensors, said sensors enabled to be individually configured by a remote medical center server.

41. The method of claim 40, wherein said array of sensors includes one or more sensors selected from the group consisting of an ECG sensor, Oxygen level sensor, pulse sensor, blood pressure sensor, SpA₂ sensor, glucose level sensor, sweat sensor, skin temperature sensor, pH level sensor, external temperature sensor, air humidity level sensor, and pollution level sensor.

42. The method of claim 24, comprising remotely configuring the wireless monitoring device, by a remote medical center server.

43. The method of claim 42, wherein said remote configuration includes implementing selected software updates.

44. The method of claim 42, wherein said remote configuration includes remotely updating client software in said wireless monitoring device, by said medical center server.

45. The method of claim 42, comprising encrypting data communicated between said wireless monitoring device and said medical center server.

46. The method of claim 42 comprising authenticating data communicated between said wireless monitoring device and said medical center server.

47. The method of claim 42, comprising authenticating data and encrypting data communicated between said wireless monitoring device and said medical center server.
